(19) United States
(12) Patent Application Publication
(10) Pub. No.: US 2002/0091081 A1
(43) Pub. Date: Jul. 11, 2002

(54) Compositions and methods for inhibiting angiogenesis

(76) Inventor: Adonia E. Papathanassiu, Silver Spring, MD (US)

Correspondence Address: Adonia Papathanassiu, P.O. Box 1001, Silver Spring, MD, 20910, US

(21) Appl. No.: 09/935,145

(22) Filed: Aug. 22, 2001

Related U.S. Application Data
(60) Provisional application No. 60/227,152, filed on Aug. 22, 2000.

(51) Int. Cl. A61K 38/17; A61K 38/10

(52) U.S. Cl. 514/12; 514/14

(57) ABSTRACT

The present invention provides a treatment for mammalian diseases characterized by pathological angiogenesis. The treatment consists of administering therapeutically active dosages of peptides containing specific amino acid sequences or antibodies that bind to cell membrane antigens on the surface of rapidly dividing endothelial cells.

### CROSS-REFERENCE TO RELATED APPLICATIONS

The present nonprovisional patent application claims benefit of provisional patent application entitled “Composition and Methods for Inhibiting Angiogenesis” with filing date Aug. 22, 2000 and patent application No. 60/227,152.

### BACKGROUND OF THE INVENTION

Angiogenesis or neovasculization is the formation of new blood vessels from pre-existing capillaries via a mechanism that involves degradation of the basement membrane which surrounds the parent vessel, migration of endothelial cells through the degraded membrane, proliferation of the migrating cells, endothelial cell differentiation, and loop formation (Folkinan, J., Angiogenesis and angiogenesis inhibition: an overview, EXS., 79, 1-8 (1997)). With the exception of wound healing and menstruation, angiogenesis in adults is observed only in pathological situations such as cancer, atherosclerosis, and psoriasis, where it contributes to the progression and symptom manifestation of the disease (Folkman, J. Angiogenesis in cancer, vascular, rheumatoid and other disease, Nat. Med. 1(1), 27-31 (1995)). Other “angiogenesis-related” diseases include endometriosis, Kaposi's sarcoma and other HIV-related conditions, leukemia, scleroderma, pyogenic granuloma, myocardial angiogenesis, corneal diseases, rubeosis, neovascular glaucoma, diabetic retinopathy, macular degeneration, and retrolental fibroplasia. As used herein, the term “angiogenesis-related diseases” means pathological conditions that require endothelial cell proliferation for progression and symptom manifestation (Chappey, O., et al. Endothelial cells in culture: an experimental model for the study of vascular dysfumctions. Cell Biol. Toxicol., 12(4-6), 199-205 (1996)).

Increasing experimental evidence suggest that angiogenesis plays an essential role in cancer development. It has been observed that solid tumors neither grow beyond 1-2 mm³ nor metastasize unless they become vascularized (Folkman, J. What is the Evidence that Tumors are Angiogenesis Dependent?, J. Natl. Canc. Inst., 82, 4-6 (1990)). Formation of tumor vasculature is necessary in order to deliver nutrients and oxygen at the tumor site, thus, providing a route for tumor metastasis to distant sites. Compositions that inhibit endothelial cell proliferation and/or migration have been shown to inhibit tumor neovascularization, and to prevent tumor growth and metastasis (Eatock, M. M., et al. Tumour vasculature as a target for anticancer therapy. Cancer Treat Rev. 26(3), 191-204 (2000)). Several of these inhibitors are currently under evaluation in human clinical trials (Deplanque, G., et al. Anti-angiogenic agents: clinical trial design and therapies in development, Eur. J. Cancer, 36, 1713-1724 (2000)).

Antibodies are proteins synthesized by B lymphocytes usually in response to the presence of a foreign substance, called an antigen (Askonas, B. A. Immunoglobulin synthesis and its induction in B-lymphoid cells, Acta Endocrinol Suppl (Copenh), 194, 117-132 (1975)). Antibodies are the recognition elements of the humoral immune response, designed to lyse foreign microorganisms and infected cells via activation of the complement system. Antibodies possess specific affinity for the antigens that induced their formation and they readily complex with them to trigger complement activation. Naturally occurring antibodies consist of two heavy and two light chains linked together by disulfide bonds. Each chain comprises of domains of unique sequence responsible for antigen binding (variable domains) and domains of constant sequence involved in complement activation and mediation of antibody-dependent cellular toxicity (constant domains). Furthermore, the variable domains of light and heavy chains have similar structure with each domain comprising four somewhat conserved regions, called the framework regions (FR), and three hyper-variable regions, called complementarity determining regions (CDR). Studies have shown that CDRs determine antibody specificity (Ohno et al. Antigen-binding specificities of antibodies are primarily determined by seven residues of VH, Proc Natl Acad Sci USA, 82(9), 2945-2949 (1985)).

Antibodies produced in response to the presence of a single antigen have a common specificity but they are heterogeneous in nature, since they are derived from many different antibody-producing cells. Homogeneous or monoclonal antibodies can be produced through hybridoma cells (Galfre, G. and Milstein, C. Preparation of monoclonal antibodies: strategies and procedures, Methods Enzymol., 73(Pt B), 3-46 (1981)). The hybridoma cell method of producing large amounts of homogeneous populations of antibodies with a particular specificity has allowed the use of monoclonal antibodies as diagnostic and therapeutic agents (Milstein, C. With the benefit of hindsight, ImmunoL Today, 21(8), 359-64 (2000)).

Initially, animal-derived monoclonal antibodies had limited therapeutic value in humans due to antigenicity. The problem was solved with the production of humanized antibodies. Humanized antibodies are defined as immunoglobulin variants or fragments capable of binding to a predetermined antigen and which comprise of a FR region having substantially the amino acid sequence of a human immunoglobulin and a CDR region having substantially the amino acid sequence of a non-human immunoglobulin (Hurle, M. R. and Gross, M. Protein engineering techniques for antibody humanization, Curr. Opin. Biotechnol., 5(4), 428-33 (1994)). Humanized antibodies have been recently approved for the treatment of various diseases including cancer. Trastuzumab, a humanized antibody against HER-2 receptor, is used for the treatment of breast cancer, while Rituximab, a humanized antibody against CD20, is used for the treatment of lymphoma (Baselga, et al. Phase II study of weekly intravenous trastuzumab (Herceptin) in patients with HER2/neu-overexpressing metastatic breast cancer, Semin. Oncol., 26(4 Suppl 12), 78-83 (1999); Slamon et al. Use of chemotherapy plus a monoclonal antibody against HER2 for metastatic breast cancer that overexpresses HER2, N. Engl. J. Med., 344(11), 783-92 (2001); Byrd et al. Rituximab using a thrice weekly dosing schedule in B-cell chronic lymphocytic leukemia and small lymphocytic lymphoma demonstrates clinical activity and acceptable toxicity, J. Clin. Oncol., 19(8), 2153-64 (2001)).

### BRIEF SUMMARY OF THE INVENTION

In accordance with the present invention, compositions and methods are provided for inhibiting angiogenesis and for treating angiogenesis-related diseases.

The compositions provided herein comprise naturally occurring or synthetic peptides containing an amino acid sequence of the following motif:

PPNXₐN_{b}PX_{c}PXPXPXN or NNNPXXXXPXPPXXP (1)

where a=2-3, b=2-3, c=3-4, P is a positively charged amino acid, i.e., arginine (R) or lysine (K), N is a negatively charged amino acid, i.e., aspartic acid (D) or glutamic acid (E), and X is any amino acid.

In further embodiments, the invention provides compositions comprise antibodies that bind to peptides containing an amino-acid sequence of the previously mentioned motif (1).

The methods provided herein for treating angiogenesis-related diseases involve administering to a human or animal a composition containing therapeutic dosages of a naturally occurring protein, protein fragments, or peptides containing an amino acid sequence of the previously mentioned motif (1).

In further embodiments, the invention provides methods for treating angiogenesis-related diseases comprise administering to a human or animal a composition containing therapeutic dosages of an antibody that binds to a peptide containing an amino acid sequence of the previously mentioned motif (1).

Thus, it is an object of the present invention to provide compositions and methods for inhibiting angiogenesis.

It is another object of the present invention to provide methods and compositions for treating cancer by inhibiting tumor neovascularization.

### BRIEF DESCRIPTION OF DRAWINGS

FIG. 1 is a schematic diagram showing the sequence of peptides EP01, EP02, and EP03 containing amino acid sequences of the previously mentioned motif (1).

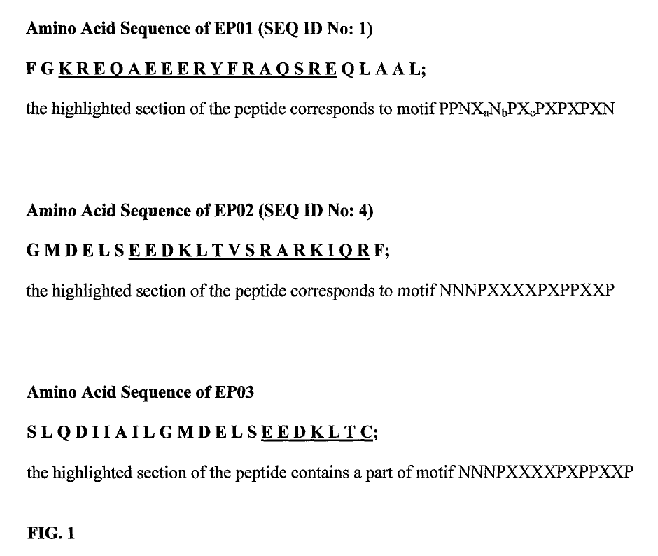

FIG. 2. is a graph depicting the ability of peptides EP01, EP02, and EP03 to inhibit basic fibroblast growth factor (bFGF)-induced proliferation of human umbilical vein endothelial cells (HUVECs). Specifically, indicated concentrations of EP01 (open squares), EP02 (open diamonds), and EP03 (open circles)

FIG. 3A and FIG. 3B are graphs depicting the ability of murine polyclonal antisera raised against peptides EP02 (anti-EP02) and EP03 (anti-EP03) and normal murine serum to specifically bind to peptides EP02 (FIG. 3A) and EP03 (FIG. 3B).

FIG. 4 is a graph depicting the ability of a murine anti-EP02 monoclonal antibody (mab), named B2G4, and a murine anti-EP03 mab, named D2G11, to specifically bind on the cell surface of HUVECs

FIG. 5 is a graph depicting the ability of B2G4 and D2G11 to inhibit bFGF-induced proliferation of HUVECs.

Compounds According to the Invention

As described below, compounds, which are useful in accordance with the invention, include naturally occurring and synthetic peptides containing an amino acid sequence of the previously mentioned motif (1) and antibodies that bind to naturally occurring and synthetic peptides containing an amino acid sequence of the previously mentioned motif (1). Synthetic peptides include but are not limited to peptides EP01 and EP02. Naturally occurring peptides include but are not limited to F₁-ATPase inhibitor protein (F₁I) and the beta (b) subunit of F₁-ATPase.

### DETAILED DESCRIPTION OF THE INVENTION

Other objects, features and aspects of the present invention are disclosed in, or are obvious from, the following Detailed Description. It is to be understood by one of ordinary skill in the art that the present discussion is a description of exemplary embodiments only and is not intended as limiting the broader aspects of the present invention, which broader aspects are embodied in the exemplary construction.

The present invention comprised of methods and compositions for treating angiogenesis-related diseases in a human or animal. The treatment comprises the administration of a peptide or antibody in sufficient amount to inhibit endothelial cell proliferation or migration and to suppress angiogenesis-related diseases.

I. Definitions

The terms “a”, “an” and “the” as used herein are defined to mean one or more and include the plural unless the context is inappropriate.

The term “peptides” relates to chains of amino acids whose alpha carbons are linked through peptide bonds formed by a condensation reaction between the carboxyl group of the alpha carbon of one amino acid and the amino group of the alpha carbon of another amino acid. A peptide has two terminal amino acids, one amino acid with a free amino-group called the amino- or N-terminus and one amino acid with a free carboxyl group called the carboxyl- or C-terminus. In a peptide, amino acids are numbered starting at the amino terminus and increasing in the direction of the carboxyl-terminus.

Peptides are produced chemically or recombinant. Solid phase is the preferred method for chemical synthesis of peptides. It involves the attachment of the C-terminal amino acid to an insoluble support and the sequential addition of the remaining amino acids. An alternative method for synthesizing amino acids is the recombinant nucleic acid method, which involves the generation of a nucleic acid sequence encoding the peptide, followed by the expression of the peptide in a host and isolation and purification of the expressed peptide.

The term “antibody” refers to monoclonal, polyclinic, multispecific (formed from at least two intact antibodies), or humanized antibodies as well as antibody fragments so long as they possess the desired biological activity. Monoclonal antibodies are obtained through the hybridoma method or the recombinant DNA method, or isolated from phage display antibody libraries. Techniques for antibody production through the previously mentioned methodologies are known to those skilled in the art. Mutlispecific or chimeric antibodies are prepared using synthetic proteins methods known in the art. Humanization of an antibody can be achieved by substituting non-human CDRs for the corresponding sequences of a human antibody as described by Jones et al., Nature, 321:522-525 (1986) and Riechmann et al., Nature, 332:323-327 (1988). Antibody fragments can be produced via proteolytic digestion or recombinant methods known in the art.

As used herein, the term “angiogenesis-related” diseases refer to pathological situations that require formation of new blood vessels for progression and symptom manifestation. Such diseases include, but are not limited to, cancer (solid tumor and leukemias), glanulomas, abnormal wound healing, atherosclerosis, rheumatoid arthritis, psoriasis, diabetic retinopathy, macular degeneration, endometriosis, and Kaposi's sarcoma, diabetic neovascularization, peptic ulcer, and scleroderma.

II. Suitable Methods for Practicing the Invention

Inhibition of Endothelial Cell Proliferation

Antiangiogenic activity is evaluated by testing the ability of a peptide or an antibody to inhibit endothelial cell growth in vitro. An endothelial cell proliferation assay typically involves the routine culturing of the endothelial cells to confluency in the appropriate media. Subsequently, the cells are trypsinized and plated in a 96-well plate at 5,000 cell per well. The cells are cultured for 96 hours in the present of the peptide or antibody and growth factors. Cell proliferation is then determined using a spectrophotometry (MTT assay, BrdU assay) o fluorimetry (Cyquant assay).

Inhibition of Tumor Growth

Ability to inhibit angiogenesis-related diseases is evaluated by testing the ability of a peptide or an antibody to suppress tumor growth in vivo. In a primary tumor growth assay, a certain number of tumor cells such as B16 melanoma cells are injected subcutaneously in C57/J6 mice. The tumor cells are allowed to grow; treatment is initiated when the tumors become palpable. Tumor size is measured every day or every other day. The experiment is terminated at a predetermined time point.

Administration

The compositions described previously may be administered by the topical, oral, rectal or parenteral (e.g., intravenous, subcutaneous or intramuscular) route. They may also be incorporated into biodegradable polymers for sustained release implanted at the disease site. The dosage of the compositions depends on the condition treated, the activity of the drug used, the route of administration, and other clinical factors such as severity of the disease and weight of the patient. The compositions are formulated in ways suitable for the specific route of administration. Formulations suitable for oral administration include capsules, cachets or tablets containing a predetermined amount of the active ingredient, powder or granules, solutions, suspensions, and emulsions. Formulations suitable for topical administration in the mouth include lozenges, pastilles, and mouthwashes. Formulations suitable for topical administration to the skin include ointments, creams, gels, pastes, and transdermal patches. Formulations for rectal administration may be presented as a suppository with a suitable base, while vaginal administrations maybe presented as pessaries, tampons, creams, gels, pastes, foams, and sprays comprising the active ingredient in an appropriate carrier. Formulations suitable for parenteral administration include aqueous and non-aqueous sterile injection solutions presented in unit-dose or multi-dose containers. It should be also understood that, in addition to the ingredients mentioned above, formulations of this invention might include other agents conventional in the art having regard to the type of formulation in question.

The invention is further understood by the following non-limiting examples, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

### EXAMPLE 1

Effect of EP01, EP02, and EP03 peptides on the bFGF-induced Proliferation of HUVECs.

Proliferation assays familiar to those skilled in the art using human umbilical vein endothelial cells (HUVECs) were employed in order to determine the effect of various antibiotics on the growth of bFGF-stimulated HUlVECs.

Materials and Methods

The materials for this experiments included endothelial cells (HUVECs) and media for their proliferation (Media 200, fetal bovine serum (FBS), gelatin, bFGF) (Paragon Bioservices, Baltimore, Md.), and Cell Titer 96 for detection of cell proliferation (Paragon Bioservices, Baltimore, Md.). Peptides EP01, EP02, and EP03 were synthesized by Multiple Peptide Systems (San Diego, Calif.).

HUVECs were routinely cultured to confluency in Media 200 containing 10% FBS. The cells were then trypsinized and plated in a 96-well plate pre-coated with 1% gelatin at 5000 cells per well per 100 μL Media 200 containing 2% FBS. The cells were allowed to adhere for 24 hours. Subsequently, the media were aspirated and fresh Media 200 containing 0.5% FBS were added to the wells followed by the addition of various concentrations of peptides in the presence and absence of 20 ng/ml bFGF followed. The assay plates were incubated at 37° C., 5% CO₂ for 48 hours. At the end of the incubation period, cell proliferation was determined using cell counting (Cell Counter Model Z1, Coulter Incorporation, Miami, Fla.) or spectrophotometry. In the later case, the assay plates were incubated with Cell Titer 96 for 2 hours and the absorbance was recorded at 490 nim. The effect of the various peptides on the proliferation of endothelial cells was expressed as % inhibition. % Inhibition is defined by the following formula:

$$\frac{\begin{matrix}[ \text{absorbance of cells treated with bFGF} ] - \\ [ absorbance of cells treated with bFGF and peptide ]\end{matrix}}{\begin{matrix}[ \text{absorbance of cells treated with bFGF} ] - \\ [ \text{absorbance of untreated cells} ] -\end{matrix}} \times 100 = \% \text{Inhibition of Proliferation}$$

Results

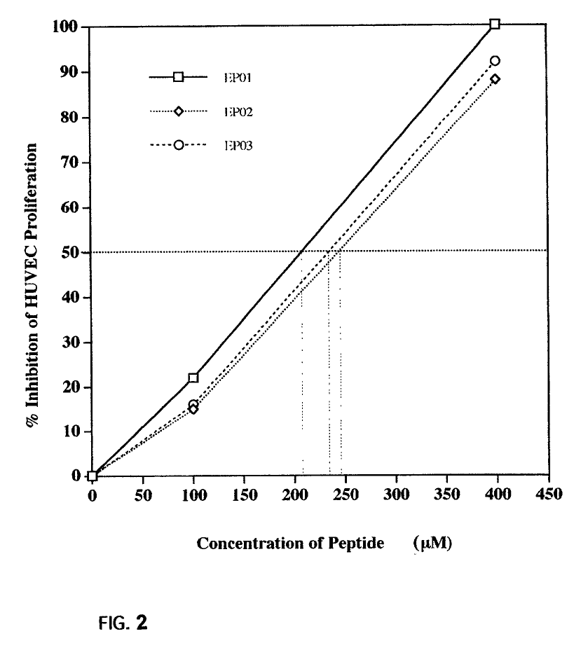

Peptides EP01, EP02, and EP03 tested here inhibited bFGF-induced HUVEC proliferation. The relative antiproliferative effects of EP01, EP02, and EP03 are shown graphically in FIG. 2. For each point of FIG. 2, the error is less than 10%. %Inhibition is defined in Materials and Methods. The IC₅₀ of the antiproliferative effects of the peptides are reported below:

| Peptide | IC₅₀ of Antiproliferative Effect |
|---|---|
| EP01 | 210 μM |
| EP02 | 235 μM |
| EP03 | 245 μM |

### EXAMPLE 2

Production of murine polyclonal antisera that bind EP02 and EP03.

Antibody production protocols familiar to those skilled in the art were employed in order to produce murine polyclonal sera, which recognize and bind to peptides with specific amino acid sequences.

Materials and Methods

Peptides EP02 and EP03 were conjugated with KLH, a highly immunogenic copper-containing protein, using a commercially available kit (Pierce, product number 77622). The resulting conjugated peptides were used for immunization of mice. After two booster immunizations, the mice were bled and murine anti-EP02 and anti-EP03 antisera were obtained. Various dilutions of these antisera were tested for their ability to bind 96-well plates coated with 2 μg/ml EP02 and EP03. Specifically, 96-well plates were incubated for 2 hrs at room temperature with 50 μl per well of either 2 μg/ml EP02 or 2 μg/ml EP03 in 50 mM Carbonate-Bicarbonate buffer, pH 9.6 (Sigma, St. Louis, Mo.). Subsequently, the wells were emptied and non-specific binding was blocked with 200 μl of 3% non-fat dry milk in PBS (BioWhittaker, Md.) (30 minutes, room temperature). The wells were washed three times with 300 μl PBS containing 0.1% Tween-20. A volume of 50 μl of polyclonal antisera diluted in PBS-0.1% Tween-20 was then added to the wells. After a 60 min incubation at room temperature, the wells were emptied and washed. This was followed by the addition of 50 μl of secondary antibody (goat anti-mouse IgG and IgM peroxidase-labeled abs) diluted in 200 μI PBS containing 0.1% Tween-20. After a 30-min incubation at room temperature, the wells were washed and 50 μl of a peroxidase substrate (ABTS, Kirkegaard and Perry) were added. Binding was measured at 405 nm.

Results

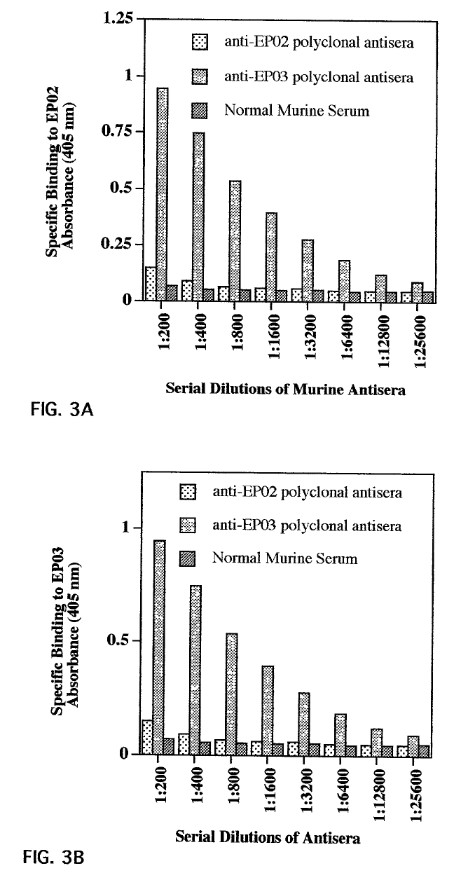

Both peptides were shown to be highly immunogenic as shown in FIG. 3. There was no cross-reactivity between the different antisera.

### EXAMPLE 3

Production of monoclonal antibodies, which recognize and bind EP02 and EP03.

Monoclonal antibody production protocols familiar to those skilled in the art were employed in order to produce monoclonal abs, which recognize and bind to peptides with specific amino acid sequences

Materials and Methods.

Monoclonal antibodies (B2G4 and D2G11), which recognize and bind EP02 and EP03 respectively, were generated from previously produced antisera according to well-known methods of antibody production (Seon et al. Monoclonal antibody that defines a unique human T-cell leukemia antigen, Proc. Natl. Acad. USA, 80, 845-849 (1983)). B2G4 and D2G11 mabs specifically recognized EP02 and EP03, respectively. These abs were also able to bind to the cell surface of HUVECs, as measured by a cell-based binding assay. Spcifically, HUVECs were plated at 75% confluency in 96-well plates and stimulated with 2 ng/ml bFGF. After overnight incubation, the wells were emptied and washed with cold PBS. This was followed by addition of 200 μl of the binding buffer (10 mM MOPS pH 6.7 containing 250 mM sucrose and 0.4 mM ATP). The cells were then incubated with 20 μg of ab for 2 hrs at 37° C. Subsequently, the wells were emptied and washed. After incubation with a fluorescein-labeled secondary ab for 30 min at 37° C., specific binding is measured with a fluorometer.

Results

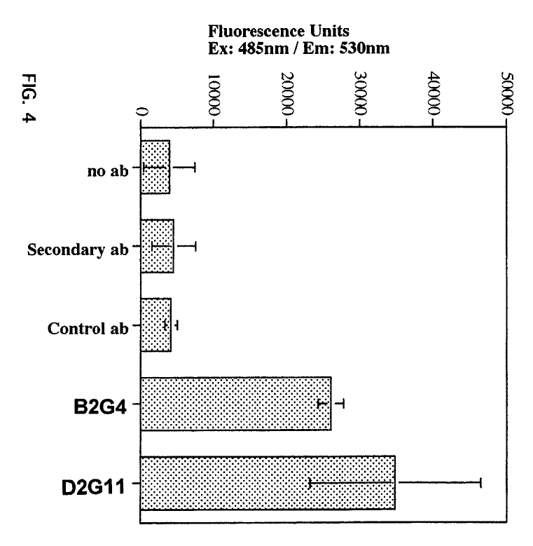

Monoclonal abs that recognize and bind EP02 and EP03 bind to the cell surface of proliferating HUVECs, as depicted in FIG. 4.

### EXAMPLE 4

Monoclonal abs B2G4 and D2G11 inhibit bFGF-induced proliferation of HUVECs.

Materials and Methods

HUVEC proliferation assays in the presence of abs were performed as previously described.

Results.

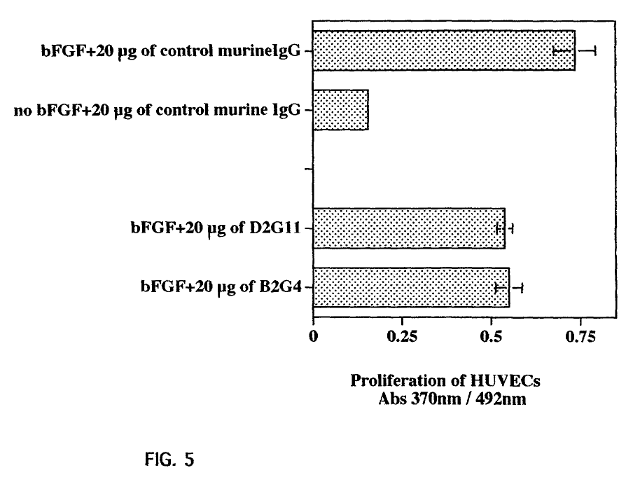

B2G4 and D2G11 mabs induce significant inhibition of bFGF-induced proliferation of HUVECs, as depicted in FIG. 5.

I claim:

1. A method of inhibiting angiogenesis in a human or animal having an angiogenesis-related disease comprising administering to the human or animal a therapeutically active dosage of a composition comprising a peptide, protein or protein fragment containing the following amino-acid sequence motif:
PPNXₐN_{b}PX_{c}PXPXPXN
where a=2-3, b=2-3, c=3-4, P is a positively charged amino acid such as arginine (R) or lysine (K), n is a negatively charged amino acid such as aspartic acid (D) or glutamic acid (E), and X is any amino acid.

2. The method of claim 1 wherein the peptide, protein, or protein fragment contains an amino-acid sequence selected from the group consisting of SEQ ID No 1, 2, and 3.

3. The method of claim 2 wherein the protein is the F₁-ATPase inhibitor protein (F₁I) (SEQ ID No 5).

4. The method claim 1 wherein the angiogenesis-related disease is selected from the group consisting of cancer, endometriosis, Kaposis's sarcoma, arthritis, psoriasis, macular degeneration, and diabetic retinopathy.

5. The method of claim 4 wherein the peptide, protein, or protein fragment contains an amino-acid sequence selected from the group consisting of SEQ ID No 1, 2, and 3.

6. The method of claim 5 wherein the protein is the F₁-ATPase inhibitor protein (F₁I) (SEQ ID No 5).

7. A method of treating a human or animal having an angiogenesis-related disease comprising administering to the human or animal a therapeutically active dosage of a composition comprising a peptide, protein or protein fragment containing the following amino-acid sequence motif:
NNNPXXXXPXPPXXP,
where P is a positively charged amino acid such as arginine (R) or lysine (K), N is a negatively charged amino acid such as aspartic acid (D) or glutamic acid (E), and X is any amino acid.

8. The method of claim 7 wherein the peptide, protein, and protein fragment contains an amino-acid sequence consisting of SEQ ID No 4.

9. The method of claim 8 wherein the protein is the beta (β) subunit of F₁-ATPase (SEQ ID No: 6).

10. The method of claim 7 wherein the angiogenesis-related disease is selected from the group consisting of cancer, endometriosis, Kaposis's sarcoma, arthritis, psoriasis, macular degeneration, and diabetic retinopathy.

11. The method of claim 10 wherein the peptide, protein or protein fragment consists of an amino-acid sequence containing SEQ ID No. 4.

12. The method of claim 11 wherein the protein is the beta (β) subunit of F₁-ATPase (SEQ ID No: 6).

13. A method of inhibiting angiogenesis in a human or animal having an angiogenesis-related disease comprising administering to the human or animal a therapeutically active dosage of a composition comprising an antibody that binds to an amino-acid sequence of the following motifs
PPNXₐN_{b}PX_{c}PXPXPXN or NNNPXXXXPXPPXXP
where a=2-3, b=2-3, c=3-4, P is a positively charged amino acid such as arginine (R) or lysine (K), N is a negatively charged amino acid such as aspartic acid (D) or glutamic acid (E), and X is any amino acid.

14. The method of claim 13 wherein the antibody is a conjugated to a cytotoxic moiety.

15. The method of claim 14 wherein the cytotoxic moiety is an antimitotic agent such as a steroid, antibiotic, antimetabolite, anthracycline, or an alkylating agent.

16. The method of claim 14 wherein the cytotoxic moiety is a plant-, fungus-, or bacteria-derived toxin such as A chain toxin, aspergillin, diphtheria toxin or Pseudonomas exotoxin.

17. The method of claim 13 wherein the antibody is a humanized antibody.

18. The method of claim 13 wherein the angiogenesis-related disease is selected from the group consisting of cancer, endometriosis, Kaposis's sarcoma, arthritis, psoriasis, macular degeneration, and diabetic retinopathy.

19. A composition of inhibiting angiogenesis in a human or animal having an angiogenesis-related disease comprising administering to the human or animal a therapeutically active dosage of a composition comprising an antibody that binds to an amino-acid sequence of the following motif:
PPNXₐN_{b}PX_{c}PXPXPXN or NNNPXXXXPXPPXXP
where a=2-3, b=2-3, c=3-4, P is a positively charged amino acid such as arginine (R) or lysine (K), N is a negatively charged amino acid such as aspartic acid (D) or glutamic acid (E), and X is any amino acid.

20. The composition of claim 19 wherein the angiogenesis-related disease is selected from the group consisting of cancer, endometriosis, Kaposis's sarcoma, arthritis, psoriasis, macular degeneration, and diabetic retinopathy.


SEQUENCE LISTING (6 sequences)

SEQ ID NO: 1 - protein, 24 aa, Homo Sapiens
FGKREQAEEERYFRAQSREQLAAL

SEQ ID NO: 2 - protein, 24 aa, Homo Sapiens
FGKREQAEEERYFRARAKEQLAAL

SEQ ID NO: 3 - protein, 24 aa, Homo Sapiens
FVKRERATEDFFVRQREKEQLRHL

SEQ ID NO: 4 - protein, 22 aa, Homo Sapiens
GMDELSEEDKLTVSRARKIQRF

SEQ ID NO: 5 - protein, 81 aa, Homo Sapiens
GSDQSENVDRGAGSIREAGGAFGKREQAEEERYFRAQSREQLAALKKHHEEEIVHHKKEIERLQKEIERHKQKIKMLKHDD

SEQ ID NO: 6 - protein, 539 aa, Homo Sapiens
MTSLWGKGTGCKLFKFRVAAAPASGALRRLTPSASLPPAQLLLRAVRRRSHPVRDYAAQTSPSPKAGAATGRIVAVIGAVVDVQFDEGLPPILNALEVQGRETRLVLEVAQHLGESTVRTIAMDGTEGLVRGQKVLDSGAPIKIPVGPETLGRIMNVIGEPIDERGPIKTKQFAPIHAEAPEFMEMSVEQEILVTGIKVVDLLAPYAKGGKIGLFGGAGVGKTVLIMELINNVAKAHGGYSVFAGVGERTREGNDLYHEMIESGVINLKDATSKVALVYGQMNQPPGARARVALTGLTVAEYFRDQEGQDVLLFIDNIFRFTQAGSEVSALLGRIPSAVGYQPTLATDMGTMQERITTTKKGSITSVQAIYVPADDLTDPAPATTFAHLDATTVLSRAIAELGIYPAVDPLDSTSRIMDPNIVGSEHYDVARGVQKILQDYKSLQDIIAILGMDELSEEDKLTVSRARKIQRFLSQPFQVAEVFTGHMGKLVPLKETIKGFQQILAGEYDHLPEQAFYMVGPIEEAVAKADKLAEEHSS